(19) United States
(12) Patent Application Publication
(10) Pub. No.: US 2023/0301879 A1
(43) Pub. Date: Sep. 28, 2023

(54) OIL-IN-WATER EMULSION COMPOSITION

(71) Applicant: SHISEIDO COMPANY, LTD., Tokyo (JP)

(72) Inventors: Ryo FURUKAWA, Tokyo (JP); Ayumu ENOMOTO, Tokyo (JP)

(73) Assignee: SHISEIDO COMPANY, LTD., Tokyo (JP)

(21) Appl. No.: 18/020,464

(22) PCT Filed: Sep. 8, 2021
(86) PCT No.: PCT/JP2021/032954

§ 371 Date: Feb. 9, 2023

(30) Foreign Application Priority Data
Sep. 9, 2020 (JP) 2020-151108

(51) Int. Cl. A61K 8/06; A61K 8/86; A61K 8/34; A61Q 17/04

(52) U.S. Cl. CPC A61K 8/062; A61K 8/86; A61K 8/345; A61Q 17/04

(57) ABSTRACT

An oil-in-water emulsion composition is to be excellent in emulsion stability even in polymer emulsification and to provide a further moist feel in use.

An oil-in-water emulsion composition is made to include (A) a (meth)acrylic acid/alkyl (meth)acrylate/(meth)acrylic acid-POE monoalkyl ether ester copolymer, (B) batyl alcohol, and (C) 10 mass % or more of a polar oil with respect to the total amount of the composition.

### TECHNICAL FIELD

The present invention relates to an oil-in-water emulsion composition.

### BACKGROUND ART

Water-in-oil emulsion cosmetics generally contain an ultraviolet absorber blended in their oil phases, and therefore have a high sunscreen effect and excellent stability. However, many water-in-oil emulsion cosmetics are difficult to wash away easily with a normal cleanser or soap, and in addition, some of them have a problem of a feel in use, such as dryness due to an ultraviolet absorber contained in a large amount. Meanwhile, oil-in-water emulsion cosmetics are advantageous in that they can be washed away relatively easily and are less likely to cause dryness.

For example, Patent Literature 1 describes an oil-in-water emulsion cosmetic in which a (meth)acrylic acid/alkyl (meth)acrylate/(meth)acrylic acid-POE monoalkyl ether ester copolymer is blended to provide a uniquely soft texture in use.

### CITATION LIST

### Patent Literature

Patent Literature 1: WO 2018/221606 A

### SUMMARY OF THE INVENTION

### Object of the Invention

In general, in a so-called polymer emulsification system in which a polymer is blended as an emulsifier, an ultraviolet-protective agent is difficult to blend stably. In the cosmetic described in Patent Literature 1, a higher alcohol blended forms, together with a non-ionic surfactant and water, an aggregate (also referred to as “α-gel”) having a lamellar liquid-crystal structure and thus an ultraviolet-protective agent is stably blended, but further improvement in emulsion stability and a further moist feel in use are desired.

The present invention has been made in view of the above circumstances, and an object of the present invention is to provide an oil-in-water emulsion composition that is excellent in emulsion stability even in polymer emulsification and provides a further moist feel in use.

### Solution to Problem

The oil-in-water emulsion composition of the present invention includes
(A) a (meth)acrylic acid/alkyl (meth)acrylate/(meth)acrylic acid-POE monoalkyl ether ester copolymer,
(B) batyl alcohol, and
(C) 10 mass % or more of a polar oil with respect to the total amount of the composition.

The oil-in-water emulsion composition of the present invention preferably further includes (D) a higher alcohol.

The oil-in-water emulsion composition of the present invention preferably further includes (E) an alkyl-modified carboxyvinyl polymer.
(C) The polar oil is preferably an ultraviolet absorber.

The mass ratio of (B) the batyl alcohol to (D) the higher alcohol is preferably 1:1 to 1:3.

In a case where the oil-in-water emulsion composition of the present invention includes a surfactant, the surfactant is preferably (F) a nonionic surfactant having a hydrophilic-lipophilic balance (HLB) of 10 to 20, and the amount of the nonionic surfactant blended is preferably 0.6 mass % or less with respect to the total amount of the composition.

### Advantageous Effects of the Invention

The oil-in-water emulsion composition of the present invention includes (A) a (meth)acrylic acid/alkyl (meth)acrylate/(meth)acrylic acid-POE monoalkyl ether ester copolymer, (B) batyl alcohol, and (C) 10 mass % or more of a polar oil with respect to the total amount of the composition, and thus can be excellent in emulsion stability and provide a further moist feel in use.

### BEST MODE FOR CARRYING OUT THE INVENTION

Hereinafter, the present invention will be described in detail.

The oil-in-water emulsion composition of the present invention includes
(A) a (meth)acrylic acid/alkyl (meth)acrylate/(meth)acrylic acid-POE monoalkyl ether ester copolymer,
(B) batyl alcohol, and
(C) 10 mass % or more of a polar oil with respect to the total amount of the composition. Hereinafter, each component will be described in detail.

In the present description, POE stands for polyoxyethylene, POP stands for polyoxypropylene, PEG stands for polyethylene glycol, and DPG stands for dipropylene glycol.

### (A) (Meth)acrylic Acid/Alkyl (Meth)acrylate/(Meth)acrylic Acid-POE Monoalkyl Ether Ester Copolymer

(A) The (meth)acrylic acid/alkyl (meth)acrylate/(meth)acrylic acid-POE monoalkyl ether ester copolymer (hereinafter, also simply referred to as component (A)) is a copolymer of
(a1) acrylic acid or methacrylic acid,
(a2) an alkyl acrylate or alkyl methacrylate, and
(a3) an ester of acrylic acid or methacrylic acid with a polyoxyethylene alkyl ether.

Examples of these ingredients include, under the names listed in the International Cosmetic Ingredient Dictionary (ICID), an acrylates/ceteth-20 methacrylate copolymer, an acrylates/steareth-20 methacrylate copolymer, an acrylates/steareth-25 methacrylate copolymer, an acrylates/steareth-50 methacrylate copolymer, an acrylates/beheneth-25 methacrylate copolymer, an acrylates/steareth-20 methacrylate crosspolymer, and an ammonium acryloyldimethyltaurate/beheneth-25 methacrylate crosspolymer, which are commercially available as aqueous dispersions (polymer emulsions).

The component (A) used in the oil-in-water emulsion composition of the present invention is preferably selected from among an acrylates/steareth-20 methacrylate copolymer (Aculyn 22 by Rohm and Haas), an acrylates/steareth-25 methacrylate copolymer (Aculyn 28 by Rohm and Haas), an acrylates/steareth-20 methacrylate crosspolymer (Aculyn 88 by Rohm and Haas), and an ammonium acryloyldimethyltaurate/beheneth-25 methacrylate crosspolymer (Aristoflex HMB by Clariant Production UK Ltd.). Among these, an acrylates/steareth-20 methacrylate copolymer or an acrylates/steareth-20 methacrylate crosspolymer is particularly preferably used.

The aqueous dispersion of the above-described acrylic acid-based polymer is diluted with water or the like as necessary, and neutralized by adding an alkaline agent to increase the viscosity. The alkaline agent used for neutralization of the copolymer is not particularly limited, and an inorganic base such as sodium hydroxide or potassium hydroxide, or an organic base such as triethanolamine, isopropanolamine, or a basic amino acid can be used.

The amount of the component (A) blended is preferably 0.01 to 3.0 mass %, more preferably 0.05 to 2.0 mass %, still more preferably 0.1 to 1.4 mass %, and still even more preferably 0.2 to 1.0 mass % in terms of the actual content of the polymer with respect to the total amount of the composition. If the amount of the component (A) blended is 0.01 mass % or more, emulsion stability can be further obtained, and if the amount is 3.0 mass % or less, the usability can be further enhanced such that the composition disintegrates in a moist state during application.

### (B) Batyl Alcohol

Batyl alcohol (C₂₁H₄₄O₃) is also referred to as octadecyl glyceryl ether, glyceryl monostearyl ether, or the like, and is a glyceryl monoalkyl ether in which a long chain aliphatic alcohol having 18 carbon atoms is ether-linked to the sn-1 position of glycerol. (B) Batyl alcohol can improve the stability of polymer emulsification of the component (A).

The amount of (B) the batyl alcohol blended is preferably 0.02 to 3 mass %, and more preferably 0.1 to 1.0 mass % with respect to the total amount of the composition. If the amount of (B) the batyl alcohol blended is 0.02 mass % or more, the emulsion stability can be further enhanced. If the amount is 3 mass % or less, moist feel in use can be obtained without a sticky feel

### (C) Polar Oil

The oil-in-water emulsion composition of the present invention includes 10 mass % or more of (C) the polar oil with respect to the total amount of the composition. (C) The polar oil is not particularly limited as long as it is usually used in cosmetics, pharmaceuticals, and foods. The IOB value is not particularly limited, but is preferably 0.05 to 0.80.

The term “IOB value” is an abbreviation of inorganic/organic balance, and represents a ratio of an inorganic value to an organic value to serve as an index indicating the degree of polarity of an organic compound. The IOB value is specifically expressed as

IOB value=inorganic value/organic value. Here, regarding the “inorganic value” and the “organic value”, one “inorganic value” or “organic value” is assigned to each atom or functional group such as a carbon atom having an “organic value” of 20 or a hydroxy group having an “inorganic value” of 100 in one molecule, and the IOB value of an organic compound can be calculated by summing the “inorganic values” and the “organic values” of all atoms and functional groups in the organic compound (see, for example, “Areas of Chemistry” by Fujita, 1957, Vol. 11, No. 10, pp.719-725).

The amount of (C) the polar oil blended is more preferably 12 to 40 mass %, and still more preferably 15 to 30 mass % with respect to the total amount of the composition. A polar oil generally tends to worsen the stability of an oil-in-water emulsion composition, but in the oil-in-water emulsion composition of the present invention, (C) the polar oil can be stably blended even if the polar oil is included at a content of 10 mass % or more with respect to the total amount of the composition.

Representative examples of the polar oil include ester oils and ultraviolet absorbers.

Specific examples of the ester oils include tripropylene glycol dineopentanoate, isononyl isononanoate, isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, cetyl ethylhexanoate, ethylene glycol di-2-ethylhexanoate, dipentaerythritol fatty acid ester, N-alkylglycol monoisostearate, neopentyl glycol dicaprate, diisostearyl malate, glycerin di-2-heptylundecanoate, trimethylolpropane tri-2-ethylhexanoate, trimethylolpropane triisostearate, pentaerythrityl tetra-2-ethylhexanoate, triethylhexanoin (glycerin tri-2-ethylhexanoate), glycerin trioctanoate, glycerin triisopalmitate, trimethylolpropane triisostearate, cetyl 2-ethylhexanoate, 2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamate-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate.

The ultraviolet absorber is not particularly limited, and is exemplified by a wide range of ultraviolet absorbers used in cosmetics generally. Examples of the ultraviolet absorbers include benzoic acid derivatives, salicylic acid derivatives, cinnamic acid derivatives, dibenzoylmethane derivatives, β,β-diphenylacrylate derivatives, benzophenone derivatives, benzylidene camphor derivatives, phenylbenzoimidazole derivatives, triazine derivatives, phenylbenzotriazole derivatives, anthranil derivatives, imidazoline derivatives, benzalmalonate derivatives, and 4,4-diarylbutadiene derivatives. Specific examples, trade names, and the like are listed below, but the ultraviolet absorbers are not limited thereto.

Examples of the benzoic acid derivatives include ethyl para-aminobenzoate (PABA), ethyl-dihydroxypropyl PABA, ethylhexyl-dimethyl PABA (for example, “Escalol 507” by ISP), glyceryl PABA, PEG-25-PABA (for example, “Uvinul P25” by BASF), and hexyl diethylamino hydroxybenzoyl benzoate (for example, “Uvinul A Plus”).

Examples of the salicylic acid derivatives include homosalate (“Eusolex HMS” by Rona/EM Industries), ethylhexyl salicylate (octyl salicylate, for example, “Neo Heliopan OS” by Haarmann & Reimer), dipropylene glycol salicylate (for example, “Dipsal” by Scher), and TEA salicylate (for example, “Neo Heliopan TS” by Haarmann & Reimer).

Examples of the cinnamic acid derivatives include octylmethoxy cinnamate or ethylhexyl methoxycinnamate (for example, “Parsol MCX” by Hoffman-La Roche), isopropyl methoxycinnamate, isoamyl methoxycinnamate (for example, “Neo Heliopan E1000” by Haarmann & Reimer), cinnoxate, DEA methoxycinnamate, diisopropyl methyl cinnamate, glyceryl-ethylhexanoate-dimethoxycinnamate, and di-(2-ethylhexyl)-4′-methoxybenzalmalonate.

Examples of the dibenzoylmethane derivatives include 4-tert-butyl-4′-methoxydibenzoylmethane (for example, “Parsol 1789”).

Examples of the β,β-diphenylacrylate derivatives include octocrylene (for example, “Uvinul N 539 T” by BASF).

Examples of the benzophenone derivatives include benzophenone-1 (for example, “Uvinul 400” by BASF), benzophenone-2 (for example, “Uvinul D 50” by BASF), benzophenone-3 or oxybenzone (for example, “Uvinul M 40” by BASF), benzophenone-4 (for example, “Uvinul MS 40” by BASF), benzophenone-5, benzophenone-6 (for example, “Helisorb 11” by Norquay), benzophenone-8 (for example, “Spectra-Sorb UV-24” by American Cyanamid), benzophenone-9 (for example, “Uvinul DS-49” by BASF), and benzophenone-12.

Examples of the benzylidene camphor derivatives include 3-benzylidene camphor (for example, “Mexoryl SD” by Chimex), 4-methylbenzylidene camphor, benzylidene camphor sulfonic acid (for example, “Mexoryl SL” by Chimex), camphor benzalkonium methosulfate (for example, “Mexoryl SO” by Chimex), terephthalylidene dicamphor sulfonic acid (for example, “Mexoryl SX” by Chimex), and polyacrylamide methylbenzylidene camphor (for example, “Mexoryl SW” by Chimex).

Examples of the phenylbenzoimidazole derivatives include phenylbenzoimidazole sulfonic acid (for example, “Eusolex 232” by Merck) and disodium phenyldibenzimidazole tetrasulfonate (for example, “Neo Heliopan AP” by Haarmann & Reimer).

Examples of the triazine derivatives include bisethylhexyloxyphenol methoxyphenyltriazine (for example, “Tinosorb S” by Ciba Specialty Chemicals), ethylhexyl triazone (for example, “Uvinul T 150” by BASF), diethylhexyl butamido triazone (for example, “Uvasorb HEB” by 3V Sigma), 2,4,6-tris(diisobutyl-4′-aminobenzalmalonate)-s-triazine, and 2,4,6-tris[4-(2-ethylhexyloxycarbonyl)anilino]-1,3,5-triazine.

Examples of the phenylbenzotriazole derivatives include drometrizole trisiloxane (for example, “Silatrizole” by Rhodia Chimie) and methylene bis(benzotriazolyl tetramethylbutylphenol) (for example, “Tinosorb M” by Ciba Specialty Chemicals).

Examples of the anthranil derivatives include menthyl anthranilate (for example, “Neo Heliopan MA” by Haarmann & Reimer).

Examples of the imidazoline derivatives include ethylhexyl dimethoxybenzylidene dioxoimidazoline propionate.

Examples of the benzalmalonate derivatives include polyorganosiloxanes having a benzalmalonate functional group (for example, polysilicone-15, “Parsol SLX” by DSM Nutrition Japan).

Examples of the 4,4-diarylbutadiene derivatives include 1,1-dicarboxy(2,2′-dimethylpropyl)-4,4-diphenylbutadiene.

The polar oil preferably contains an ultraviolet absorber, and the entire of the polar oil may be an ultraviolet absorber. The ultraviolet absorbers may be blended singly or in combination of two or more kinds thereof.

### (D) Higher Alcohol

The oil-in-water emulsion composition of the present invention preferably includes a higher alcohol. The higher alcohol is not particularly limited as long as it is a higher alcohol having 6 or more carbon atoms that can be used in the fields of cosmetics, pharmaceuticals, quasi-drugs, and the like, and examples of the higher alcohol include saturated straight chain monohydric alcohols and unsaturated monohydric alcohols.

Examples of the saturated straight chain monohydric alcohols include dodecanol (lauryl alcohol), tridodecanol, tetradodecanol (myristyl alcohol), pentadecanol, hexadecanol (cetyl alcohol), heptadecanol, octadecanol (stearyl alcohol), nonadecanol, icosanol (aralkyl alcohol), henicosanol, docosanol (behenyl alcohol), tricosanol, tetracosanol (carnaubyl alcohol), pentacosanol, and hexacosanol (ceryl alcohol).

Examples of the unsaturated monohydric alcohols include elaidyl alcohol. In the present invention, the saturated straight chain monohydric alcohols are preferable from the viewpoint of temporal stability.

As the higher alcohol in the present invention, the above-described higher alcohols can be used singly or in combination of two or more kinds thereof. In the present invention, a monohydric aliphatic alcohol having 16 to 22 carbon atoms is preferably used. In the present invention, in the case of using a plurality of higher alcohols, a mixture obtained by combination of higher alcohols such that the melting point of the mixture is 60° C. or higher is particularly preferable from the viewpoint of stability.

The amount of (D) the higher alcohol blended is preferably 0.02 to 9 mass %, more preferably 0.05 to 5.0 mass %, still more preferably 0.1 to 3.0 mass %, and particularly preferably 0.2 to 1 mass % with respect to the total amount of the composition. If the amount of (D) the higher alcohol blended is 0.02 mass % or more, the emulsion stability can be further enhanced. If the amount is 9 mass % or less, moist feel in use can be obtained without a sticky feel.

In a case where the oil-in-water emulsion composition of the present invention includes (D) the higher alcohol, the mass ratio of (B) the batyl alcohol to (D) the higher alcohol is preferably 1:1 to 1:3. In a case where (D) the higher alcohol is included, (B) the batyl alcohol and (D) the higher alcohol form an α-form hydrated crystal structure. As a result, in polymer emulsification with weak stability, the emulsion stability can be further improved, and the usability can be further enhanced such that a moist feel can be obtained without a sticky feel in use.

### (E) Alkyl-Modified Carboxyvinyl Polymer

Suitable examples of the alkyl-modified carboxyvinyl polymer include (acrylates/alkyl acrylate (C10-C30)) crosspolymers from the viewpoint of obtaining good emulsion stability and a moist feel in use. As the (acrylates/alkyl acrylate (C10-C30)) crosspolymers, a commercially available product may be used, and examples of the product include PemulenTR-2 Polymeric Emulsifier (by Lubrizol Advanced Materials, Inc.).

The amount of (E) the alkyl-modified carboxyvinyl polymer blended is preferably in a range of 0.01 to 1 mass %, more preferably in a range of 0.03 to 0.5 mass %, and still more preferably in a range of 0.03 to 0.2 mass % with respect to the total amount of the composition. If the amount of (E) the alkyl-modified carboxyvinyl polymer blended is 0.01 mass % or more, the stability can be further improved. If the amount is 1 mass % or less, further moist and light feel in use can be obtained.

### (F) Nonionic Surfactant having HLB of 10 to 20

In the oil-in-water emulsion composition of the present invention, the amount of the surfactant blended is preferably 1 mass % or less with respect to the total amount of the composition. If the amount of the surfactant blended is 1 mass % or less, the usability can be further enhanced such that a moist feel can be obtained without a sticky feel in use. In particular, the oil-in-water emulsion composition of the present invention has high emulsion stability, and therefore it may contain no surfactant. In the case of blending a surfactant according to the composition form or the like, the surfactant is preferably a nonionic surfactant having a hydrophilic-lypophilic balance (HLB) of 10 to 20. A nonionic surfactant having an HLB of 12 to 20 is more preferable. The amount of (F) the nonionic surfactant blended is preferably 0.01 to 1 mass %, more preferably 0.05 to 0.8 mass %, and still more preferably 0.1 to 0.6 mass % with respect to the total amount of the composition. If the amount of (F) the nonionic surfactant blended is 0.01 mass % or more, the surfactant can contribute to formation of an α-gel, and if the amount is 1 mass % or less, the usability without a sticky feel in use can be further enhanced.

The nonionic surfactant can be selected from among glycerin or polyglycerin fatty acid esters, propylene glycol fatty acid esters, POE sorbitan fatty acid esters, POE sorbitol fatty acid esters, POE glycerin fatty acid esters, POE fatty acid esters, POE alkyl ethers, POE alkyl phenyl ethers, POE POP alkyl ethers, POE castor oil or a hardened castor oil derivative, POE beeswax lanolin derivatives, alkanolamides, POE propylene glycol fatty acid esters, POE alkylamines, POE fatty acid amides, and the like. Specific examples of the nonionic surfactant include hydrogenated castor oil derivatives such as PEG-40 hydrogenated castor oil, PEG-50 hydrogenated castor oil, PEG-60 hydrogenated castor oil, PEG-60 glyceryl isostearate, and PEG-50 hydrogenated castor oil succinate, and ceteth-25.

The oil-in-water emulsion composition of the present invention can contain, in addition to (A) to (F) described above, an oil selected from oils, other than (C) the polar oil and (D) the higher alcohol, that are generally used in cosmetics, as long as the stability is not impaired. Examples of a desirable oil include liquid oils and fats, solid oils and fats, waxes, hydrocarbon oils, higher fatty acids, and silicone oils.

Examples of the liquid oils and fats include avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil.

Examples of the solid oils and fats include cacao butter, coconut oil, hardened coconut oil, palm oil, palm kernel oil, Japan wax kernel oil, hardened oils, Japan wax, and hardened castor oil.

Examples of the waxes include beeswax, candelilla wax, cotton wax, carnauba wax, bayberry wax, Chinese wax, montan wax, rice bran wax, kapok wax, sugar cane wax, jojoba wax, shellac wax, and POE cholesterol ether.

Examples of the hydrocarbon oils include liquid paraffin, ozokerite, squalane, pristane, paraffin, ceresin, squalene, petrolatum, microcrystalline wax, and hydrogenated polydecene.

Examples of the higher fatty acids include lauric acid, myristic acid, palmitic acid, stearic acid, behenic acid, oleic acid, undecylenic acid, tall oil acid, isostearic acid, linolic acid, linoleic acid, eicosapentaenoic acid (EPA), and docosahexaenoic acid (DHA).

Examples of the silicone oils include chain polysiloxanes (such as dimethylpolysiloxane, methylphenylpolysiloxane, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, and various modified polysiloxanes (such as an amino-modified polysiloxane, a polyether-modified polysiloxane, an alkyl-modified polysiloxane, and a fluorine-modified polysiloxane).

The oils can be used singly or in appropriate combination of two or more kinds thereof.

In the case of blending the oils other than (C) the polar oil and (D) the higher alcohol, the amount of the oils blended is preferably 1.0 to 20 mass %, and more preferably 5.0 to 15 mass % with respect to the total amount of the composition. If the amount of the oils blended is 1.0 mass % or more, precipitation of the ultraviolet absorber can be suppressed and compatibility of the oils can be ensured, and if the amount is 20 mass % or less, the emulsion stability and the usability can be further improved.

In the oil-in-water emulsion composition of the present invention, other optional components that can be blended in normal cosmetics and quasi-drugs can be blended as long as an effect of the present invention is not impaired. Examples of other optional components include, but are not limited to, powder components, coloring agents, moisturizers, aqueous thickeners, dispersants, preservatives, perfumes, and various agents.

Examples of the powder components include ultraviolet scattering agents such as zinc oxide and titanium oxide, extender pigments such as talc, mica, and kaolin, and polymer powders such as a polyethylene powder, a polymethylmethacrylate powder, and nylon powder.

Examples of the moisturizers include polyhydric alcohols such as glycerin, 1,3-butylene glycol, dipropylene glycol, and propylene glycol, and water-soluble polymers such as trehalose, hyaluronic acid, and chondroitin sulfate.

Examples of the aqueous thickeners include succinoglycan, a (dimethylacrylamide/acryloyldimethyltaurine Na) cross polymer, cellulose gum, a carboxyvinyl polymer, xanthan gum, and a (hydroxyethyl acrylate/acryloyldimethyltaurine Na) copolymer.

The oil-in-water emulsion composition of the present invention can be produced in accordance with a common method for producing an oil-in-water emulsion composition (cosmetic) such as a method in which components to be included in an oil phase and components to be included in an aqueous phase are separately mixed, the oil phase is added to the aqueous phase, and the mixture is emulsified.

The oil-in-water emulsion composition of the present invention can be provided as cosmetics of various dosage forms such as liquid, milky, creamy, gel, and bavarois forms.

The oil-in-water emulsion composition of the present invention can be provided as a skin care cosmetic, a sunscreen cosmetic, a cosmetic in which a coloring agent is blended, a foundation, a makeup base, or a BB cream.

The oil-in-water emulsion composition of the present invention can provide a special moist feel in use by polymer emulsification in which (A) the (meth)acrylic acid/alkyl (meth)acrylate/(meth)acrylic acid-POE monoalkyl ether ester copolymer is used, and in addition, the emulsion stability is enhanced by using (B) the batyl alcohol in combination, and therefore (C) the polar oil typified by an ultraviolet absorber can be blended at a content of 10% or more. Therefore, if an ultraviolet absorber is blended as (C) the polar oil, a cosmetic can be obtained that provides a moist feel in use and has high ultraviolet protection ability and excellent emulsion stability.

### EXAMPLES

Hereinafter, the present invention will be described more specifically with reference to Examples, but the present invention is not limited thereto at all. The unit of the amount of a blended component is mass % unless otherwise specified.

### Examples 1 to 6 and Comparative Example 1

Oil-in-water emulsion compositions were prepared in accordance with the formulas shown in Table 1 below, and the prepared compositions were evaluated in accordance with the following criteria.

### (Rolling Test)

A cylindrical container was half-filled with a prepared oil-in-water emulsion composition, the sample was subjected to rolling motion at 45 rpm for 4 hours at room temperature with a rolling tester (manufactured by Nigorikawa Rika Kogyo), and the state change of the composition was observed and evaluated in accordance with the following criteria.
A: No change is observed.
B: Slight change in viscosity or slight increase of emulsified particles is observed, but there is no problem in use.
C: Significant change in viscosity or significant increase of emulsified particles is observed, and thus use is hindered.
D: Oil separation is observed.

### (Usability)

A prepared oil-in-water emulsion composition was used by 10 expert panelists to evaluate the sliminess after application. The evaluation criteria are as follows.

### <Evaluation Points>

5 points: Very good
4 points: Good
3 points: Normal
2 points: Poor
1 point: Very poor

### <Evaluation Criteria>

A: The average is 4.5 points or more.
B: The average is 3.5 points or more and less than 4.5 points.
C: The average is 2.5 points or more and less than 3.5 points.
D: The average is less than 2.5 points.

Table 1 shows the formulas and the evaluation results. The commercial product names of the main components shown in Table 1 are as follows.
(Acrylates/alkyl acrylate (C10-C30)) crosspolymer: PEMULEN TR-2 (by Lubrizol Advanced Materials, Inc.) (Ammonium acryloyldimethyltaurate/beheneth-25 methacrylate) crosspolymer: ARISTOFLEX HMB (by Clariant GMBH)
PEG-60 glyceryl isostearate: EMALEX GWIS-160 (by Nihon Emulsion Co., Ltd.)
PEG-60 hydrogenated castor oil: NIKKOL HCO-60 (by NIPPON SURFACTANT INDUSTRIES CO., LTD.)
Lauryl betaine: ANON BL-SF (by NOF CORPORATION)
Bis-butyldimethicone polyglyceryl-3: silicone KF-6109 (by Shin-Etsu Silicone)
Polysilicone-11: Gransil DMG-3 (by GRANT INDUSTRIES)

TABLE 1

| | | Example | Example | Example | Example | Example | Example | Comparative |
|---|---|---|---|---|---|---|---|---|
| | Component list | 1 | 2 | 3 | 4 | 5 | 6 | Example 1 |
| | Water | 61.88 | 61.98 | 62.18 | 62.08 | 61.48 | 61.88 | 62.18 |
| | Glycerin | 5 | 5 | 5 | 5 | 5 | 5 | 5 |
| | DPG | 5 | 5 | 5 | 5 | 5 | 5 | 5 |
| | Carbomer | 0.2 | 0.2 | 0.2 | 0.2 | 0.2 | 0.2 | 0.2 |
| (E) | (Acrylates/alkyl acrylate | 0.05 | 0.05 | 0.05 | 0.05 | 0.05 | 0.05 | 0.05 |
| | (C10-C30)) crosspolymer | | | | | | | |
| (A) | (Ammonium | 0.4 | 0.4 | 0.4 | 0.4 | 0.4 | 0.4 | 0.4 |
| | acryloyldimethyltaurate/ | | | | | | | |
| | beheneth-25 methacrylate) | | | | | | | |
| | crosspolymer | | | | | | | |
| | Xanthan gum | 0.05 | 0.05 | 0.05 | 0.05 | 0.05 | 0.05 | 0.05 |
| (F) | PEG-60 glyceryl isostearate | 0.2 | 0.2 | 0.2 | | 0.6 | | 0.2 |
| | PEG-60 hydrogenated castor oil | | | | | | 0.2 | |
| | Lauryl betaine | 0.3 | 0.3 | 0.3 | 0.3 | 0.3 | 0.3 | 0.3 |
| | Bis-butyldimethicone | 0.3 | 0.3 | 0.3 | 0.3 | 0.3 | 0.3 | 0.3 |
| | polyglyceryl-3 | | | | | | | |
| | Potassium hydroxide | 0.12 | 0.12 | 0.12 | 0.12 | 0.12 | 0.12 | 0.12 |
| (B) | Batyl alcohol | 0.3 | 0.3 | 0.3 | 0.3 | 0.3 | 0.3 | |
| (D) | Behenyl alcohol | 0.6 | | 0.3 | 0.6 | 0.6 | 0.6 | 0.6 |
| | Stearyl alcohol | | 0.5 | | | | | |
| | Isodecyl neopentanoate | 2 | 2 | 2 | 2 | 2 | 2 | 2 |
| | Dimethicone | 1.5 | 1.5 | 1.5 | 1.5 | 1.5 | 1.5 | 1.5 |
| | Polysilicone-11/dimethicone | 4 | 4 | 4 | 4 | 4 | 4 | 4 |
| | (3CS) | | | | | | | |
| (C) | Octocrylene | 5 | 5 | 5 | 5 | 5 | 5 | 5 |
| | Octyl salicylate | 5 | 5 | 5 | 5 | 5 | 5 | 5 |
| | Homosalate | 5 | 5 | 5 | 5 | 5 | 5 | 5 |
| | t- | 2 | 2 | 2 | 2 | 2 | 2 | 2 |
| | Butylmethoxydibenzoylmethane | | | | | | | |
| | Sodium pyrosulfite | 0.1 | 0.1 | 0.1 | 0.1 | 0.1 | 0.1 | 0.1 |
| | Phenoxyethanol | 1 | 1 | 1 | 1 | 1 | 1 | 1 |
| | Total | 100 | 100 | 100 | 100 | 100 | 100 | 100 |
| Evaluation | Rolling test | A | A | A | A | A | A | D |
| | Usability | A | A | A | A | B | A | A |

As shown in Table 1, in Examples 1 to 6 in which (A) the (meth)acrylic acid/alkyl (meth)acrylate/(meth)acrylic acid-POE monoalkyl ether ester copolymer, (B) the batyl alcohol, and (C) 10 mass % or more of the polar oil with respect to the total amount of the composition are included, the emulsion stability was excellent even in polymer emulsification, and a moist feel in use was obtained without sliminess after application. Meanwhile, in Comparative Example 1 in which (B) batyl alcohol was not included, no emulsion stability was obtained even when the higher alcohol was included.

1. An oil-in-water emulsion composition comprising:
(A) a (meth)acrylic acid/alkyl (meth)acrylate/(meth)acrylic acid-POE monoalkyl ether ester copolymer;
(B) batyl alcohol; and
(C) 10 mass % or more of a polar oil with respect to a total amount of the oil-in-water emulsion composition.

2. The oil-in-water emulsion composition according to claim 1, further comprising (D) a higher alcohol.

3. The oil-in-water emulsion composition according to claim 1, further comprising (E) an alkyl-modified carboxyvinyl polymer.

4. The oil-in-water emulsion composition according to claim 3, wherein (C) the polar oil is an ultraviolet absorber.

5. The oil-in-water emulsion composition according to claim 2, wherein a mass ratio of (B) the batyl alcohol to (D) the higher alcohol is 1:1 to 1:3.

6. The oil-in-water emulsion composition according to claim 1, wherein (F) a nonionic surfactant having a hydrophilic-lipophilic balance (HLB) of 10 to 20 is contained at a content of 0.6 mass % or less with respect to the total amount of the oil-in-water emulsion composition.
